(19) United States
(12) Patent Application Publication
(10) Pub. No.: US 2026/0151446 A1
(43) Pub. Date: Jun. 4, 2026

(54) IMPROVED ORAL BIOAVAILABILITY FORMULATION FOR MITRAGYNA SPECIOSA (KRATOM)

(71) Applicant: UNIVERSITY OF FLORIDA RESEARCH FOUNDATION, INCORPORATED, Gainesville, FL (US)

(72) Inventors: Erin C. BERTHOLD, Gainesville, FL (US); Christopher Robert McCURDY, Gainesville, FL (US); Abhisheak SHARMA, Gaineville, FL (US); Elizabeth MAXWELL, Gaineville, FL (US)

(21) Appl. No.: 19/123,532

(22) PCT Filed: Oct. 30, 2023
(86) PCT No.: PCT/US2023/078192

§ 371 Date: Apr. 23, 2025

Related U.S. Application Data
(60) Provisional application No. 63/420,216, filed on Oct. 28, 2022.

(51) Int. Cl. A61K 36/74; A61K 9/48; A61P 19/02; A61P 25/04

(52) U.S. Cl. CPC A61K 36/74; A61K 9/4825; A61K 9/4866; A61P 19/02; A61P 25/04

(57) ABSTRACT

The present invention relates to pharmaceutical formulations for kratom extract and mitragynline, which is the major alkaloid extracted from kratom, and use methods thereof for treatment of osteoarthritis in canine subjects.

### GOVERNMENT FUNDING SUPPORT

This invention was made with government support under R01 DA047855 and UH3 DA048353 awarded by the National Institutes of Health. The government has certain rights in the invention.

### BACKGROUND

### Field of the Invention

This invention generally relates to the field of medicine and pharmaceutics. In particular, the invention provides a kratom formulation comprising mitragynine as an active ingredient and other excipients that increase the solubility and thus oral bioavailability of mitragynine.

### Background of the Invention

Osteoarthritis is a type of degenerative joint disease that results from breakdown of joint cartilage and underlying bone. The most common symptoms are joint pain, swelling, and stiffness, which progress slowly over a period of years. Osteoarthritis is believed to be caused by mechanical stress on the joint and low-grade inflammatory processes. Diagnosis is typically based on signs and symptoms, with medical imaging and other tests used to support or rule out other problems. Treatment includes exercise, decreasing joint stress, and pain medications such as acetaminophen or NSAIDs such as naproxen or ibuprofen, or in some cases, joint replacement surgery. Another alternative therapy for osteoarthritis is stem cell transplantation.

Osteoarthritis is a common problem in dogs, particularly in senior and large breed dogs. This condition results in destruction of the joints, chronic pain, and difficulty walking, sitting, or standing. No cure is available, but some treatments are available to reduce pain and inflammation in this degenerative condition, including joint supplements, NSAIDs, diet changes and physical therapies. Other oral analgesic options (especially oral opioids) available for companion animals have questionable bioavailability and variable efficacy. While kratom has been used anecdotally, commercial preparations may have low oral bioavailability and therefore lack of efficacy.

Kratom (Mitragyna speciosa) is a plant of the Rubiaceae family, related to coffee, which is native to Southeast Asia. Compounds in the plant have opioid and stimulant-like properties, but have not been approved as a therapeutic agent in the United States. Nevertheless, some people use kratom as an herbal remedy to self-medicate for pain, for opioid withdrawal symptoms, and for recreational purposes, and to treat pain in canines. Active compounds in kratom include, but are not limited to mitragynine, corynantheidine, speciogynine, speciociliatine, paynantheine, and corynoxine.

Kratom is usually consumed by preparing the leaves or dried leaves as a tea or decoction or by grinding them into a powder for consumption. Its increasing use for recreational purposes and for self-medication (without FDA approval or studies definitively showing its therapeutic effects and proper dosing to manage toxicity and dependence) creates a public health concern for humans. A major limitation when administering kratom products is the poor solubility and resulting in poor systemic exposure of powdered extracts obtained from the plant.

Kratom pharmacology is complex, since it contains a cocktail of psychoactive alkaloids (for example, mitragynine, speciociliatine, speciogynine, paynantheine, and corynantheidine), but it produces most of its effects through multiple receptors (opioid, adrenergic, and serotonin) in the brain [Leon, Francisco, et al. “Activity of Mitragyna speciosa (“kratom”) alkaloids at serotonin receptors.” Journal of Medicinal Chemistry 64.18 (2021): 13510-13523. Obeng, Samuel, et al. “Investigation of the adrenergic and opioid binding affinities, metabolic stability, plasma protein binding properties, and functional effects of selected indole-based kratom alkaloids.” Journal of medicinal chemistry 63.1 (2019): 433-439]. Mitragynine is the most abundant active alkaloid in Mitragyna speciosa. The diversity of active and possibly synergistically active alkaloids in kratom produces a physiological effect of mixed opiate-like and stimulant effects, with stimulant effects more prevalent at low doses and opioid effects more prevalent at high doses. The multi-modal effects of kratom are not well studied, and there have been reports of problematic organ dysfunction and toxicity, possible drug-drug interactions, and other concerns. Dose, dosage regimen, and formulation type are important factors when kratom is used for pain relief in both humans and canines. Accordingly, there is a need in the art for improved formulations for administration of kratom in order to deliver kratom alkaloids, in particular mitragynine, more effectively by improving solubility and oral bioavailability.

### SUMMARY OF THE INVENTION

One of the obstacles to natural product drug development is the complexity of the raw material and physiochemical properties of the active compounds. Kratom preparations contain many compounds that have a complex pharmacology.

When delivering compounds orally, the bioavailability of the compound(s) of interest is important to determine. This parameter indicates how much compound is absorbed through the gastrointestinal tract into the systemic circulation and is dependent upon dissolution, permeability, and first pass metabolism.

Studies on the bioavailability of mitragynine in rats revealed large differences depending on the formulation administered. The bioavailability of mitragynine, when delivered as a purified aqueous solution, was 17% but rose to 25% when delivered as a lyophilized tea extract, and increased further to 31% when delivered as an organic extract¹⁶. A commercial product analysis of a liquid kratom extract solution showed a 1.6-fold greater exposure of mitragynine compared to the lyophilized kratom tea preparation¹⁶.

In this disclosure, new formulations were developed as pharmaceutical compositions to improve solubility and thus bioavailability of kratom extract and mitragynine, which is the major alkaloid in kratom extract and known for its antinociceptive activity. The main objective of this disclosure is to utilize kratom extract composition or mitragynine composition for the treatment of pain, especially caused by arthritis in dogs, which would provide reliable and rigorous results.

To this end, a capsule dosage form is provided here, which comprises kratom extract powder or mitragynine as main ingredient compound and other excipients of such combinations as polyanionic beta cyclodextrin derivative (e.g., Captisol@), crospovidone (e.g., Kollidon® CL) and microcrystalline cellulose (e.g., Avicel®), or poloxamer 407 (e.g., Kolliphor® P407), colloidal silicon dioxide, Kollidon® and Avicel®. With the kratom extract composition, a randomized, double-blinded, masked, positively-controlled clinical trial was performed in client-owned dogs of any breed or sex, weighting 25 to 45 kg. As a result, kratom formulation comprising 45% mitragynine at a dose of mitragynine 0.5-0.7 mg/kg every 8 hours relieved pain in the tested dogs.

Therefore, embodiments of the present invention provide a capsule formulation for oral administration of kratom extract to deliver mitragynine more effectively by improving solubility and oral bioavailability. In a specific embodiment, provided is a kratom composition that includes:
(a) about 4% to about 30% by weight kratom extract powder comprising about 2% to about 65% mitragynine (w/w), optionally about 8% by weight kratom extract powder comprising about 45% mitragynine (w/w); or about 2% to about 10% by weight mitragynine, optionally 4% by weight mitragynine;
(b) about 2% to about 40% by weight polyanionic beta cyclodextrin derivative, optionally about 4%-16% by weight polyanionic beta cyclodextrin derivative;
(c) about 10% to about 50% by weight crospovidone, optionally about 40% by weight crospovidone; and
(d) the remaining weight is filled with an excipient, wherein the excipient is microcrystalline cellulose;
wherein the composition is for oral administration.

According to another embodiment, provided is a method of treatment for osteoarthritis in a subject canine comprising administering a composition comprising a therapeutically effective dose of kratom extract powder comprising mitragynine; a polyanionic beta cyclodextrin derivative, crospovidone and an excipient (optionally microcrystalline cellulose). In a specific embodiment, the method comprises administering a therapeutically effective amount of a composition comprising:
(a) about 4% to about 30% by weight kratom extract powder comprising about 2% to about 65% mitragynine, or freebase or salt thereof (w/w), optionally about 8% by weight kratom extract powder comprising about 45% mitragynine (w/w); or about 2% to about 10% by weight mitragynine, optionally 4% by weight mitragynine;
(b) about 2% to about 40% by weight polyanionic beta cyclodextrin derivative, optionally about 4%-16% by weight polyanionic beta cyclodextrin derivative;
(c) about 10% to about 50% by weight crospovidone, optionally about 40% by weight crospovidone; and
(d) the remaining weight is filled with an excipient, wherein the excipient is optionally microcrystalline cellulose;
wherein the composition is for oral administration.

In a further embodiment, provided is a method of treatment for post-surgical pain in a subject canine comprising administering to the subject about 0.5 mg/kg to about 0.7 mg/kg of the kratom extract formulation described above.

The foregoing and other embodiments are further described herein.

### BRIEF SUMMARY OF THE DRAWINGS

FIG. 1 shows chemical structure of various kratom alkaloids.

FIG. 2 is a graph showing in vitro dissolution profile of 4 different ratios of Captisol® to kratom extract powder comprising 45% mitragynine (w/w) at pH 1.2. Values represent the mean (N=3) ±SD.

FIG. 3 is a graph showing mean plasma concentration-time profiles of mitragynine after an oral dose of traditional kratom preparation (LKT; 5.7 mg/kg mitragynine), a commercial kratom product (OPMS; 9.6 mg/kg mitragynine), or the 45% mitragynine (w/w) kratom extract capsule formulation (kratom capsule; 8.0 mg/kg mitragynine). Values represent the mean (N=4)±SEM.

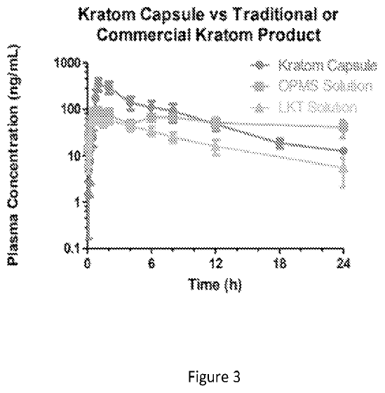

FIG. 4 is a graph showing comparison of the in vitro dissolution profile of mitragynine for the initial kratom capsule formulation versus the reformulation which contained 3× (w/w) Avicel® (microcrystalline cellulose) as a diluent (pH 1.2).

FIG. 5 is a graph showing reformulation mean plasma concentration-time profiles of mitragynine after an oral dose of kratom capsule in male and female rats. Values represent the mean (N=4) ±SEM.

FIG. 6 is a detection result of PainTrace® of Dog 1 after dose and frequency escalation, demonstrating a decline in baseline pain measures (Baseline Delta −0.66>−0.3>−0.02>0.11), denoting summative pain during exam improved.

### DETAILED DESCRIPTION

### A. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2.

As used herein, the term “kratom” refers to Mitragyna speciosa, an herbal leaf of the Rubiaceae family. The term “kratom extract” refers to a powder or semi-solid or liquid composition containing active ingredients from kratom. Kratom extracts may also include a carrier, vehicle, and/or excipient in combination with the kratom plant material.

As used herein, the term “subject” refers to any animal, and can include humans, mammalian companion animals, mammalian farm animals, and mammalian sport animals. For example, subjects include, but are not limited to simians, humans, avians, felines, canines, equines, rodents, bovines, porcines, ovines, caprines. A suitable subject for the invention preferably is suspected of having, has been diagnosed as having, or is at risk of developing a disease that can be ameliorated, treated or prevented by compositions according to embodiments of the invention. Preferred subjects are canines suffering from osteoarthritis.

As used herein, the term “administering” and its cognates refers to introducing an agent to a subject, and can be performed using any of the various methods or delivery systems for administering agents or pharmaceutical compositions known to those skilled in the art. Modes of administering include, but are not limited to oral administration or intravenous, subcutaneous, intramuscular or intraperitoneal injections, rectal administration by way of suppositories or enema, or local administration directly into or onto a target tissue, or administration by any route or method that delivers a therapeutically effective amount of the drug or composition to the cells or tissue to which it is targeted. Preferably, administration according to embodiments of this invention is oral administration.

As used herein, the terms “treatment,” “treating,” and the like, as used herein in the context of treating a disease or condition refer to obtaining a desired pharmacologic and/or physiologic effect. “Treatment,” includes: inhibiting the condition or disease or symptom thereof, such as, arresting its development or progression and also includes relieving, alleviating or ameliorating the condition or disease or one or more symptoms thereof, such as, for example, causing relief of pain, inflammation.

As used herein, the terms “excipient” and the like, in the context of pharmaceuticals, refer to any substances other than the active ingredient or agent, used in pharmaceutical dosage forms. The excipients are considered as inert substances, i.e., they do not have any active role in therapeutics, but they can be used to support the process to produce an effective product. Examples of excipients are active pharmaceutical ingredient excipients, binder excipients, capsule shell excipients, carrier excipients, coating systems excipients, controlled release excipients, diluent excipients, disintegrant excipients, effervescent system excipients, emulsifier excipients, film former excipients, flavor excipients, high-functionality excipients, lipid excipients, lubricant excipients, modified release excipients, penetration enhancer excipients, permeation enhancer excipients, pH modifier excipients, plasticizer excipients, preservative excipients, sachet filling excipients, solubilizer excipients, solvent excipients, surfactant excipients, sustained release excipients, taste masking excipients, thickener excipients, viscosity modifier excipients, blending excipients, filler excipients, compaction excipients, direct compression excipients, dry granulation excipients, hot melt extrusion excipients, wet granulation excipients, rapid release agent excipients, film formation excipients, increased bioavailability excipients, dispersion excipients, solubility enhancement excipients, stabilizer excipients, capsule filling excipients, powder blends excipients, tablet compressibility excipients, etc. (https://www.americanpharmaceuticalreview.com/25335-Pharmaceutical-Raw-Materials-and-APIs/25283-Pharmaceutical-Excipients/)

As used herein, the terms “formula,” “formulation,” and the like refer to a composition containing one or more active agents and a pharmaceutically acceptable carrier or excipient.

As used herein, the term “dosage form” refers to pharmaceutical preparations in which a specific mixture of active ingredients of a drug and inactive components (excipients) are formulated in a particular shape or form to facilitated administration and accurate delivery of active ingredients, and/or to be presented in the market. Solid dosage forms include powders, tablets, granules, capsules, cachets, pills, lozenges, suppositories. Semi-solid dosage forms include ointment, creams, paste, gels, poultices. Liquid dosage forms include collodions, droughts, elixirs, emulsions, suspension, enemas, gargles, linctuses, lotion, liniments, mouth washes, nasal drop, paints, solutions, syrups. Gaseous dosage forms include aerosols, inhalations, and sprays. (https://thepharmapedia.com/pharmaceutical-dosage-form-pharmaceutics/pharmacy-notes/).

The term “therapeutically effective amount” refers to a dose of the substance or composition that will lead to the desired pharmacological and/or therapeutic effect. The desired pharmacological effect is, to alleviate a condition or disease described herein, or symptoms associated therewith. A therapeutically effective amount of a substance may vary according to factors such as the disease state, age, sex, and weight of the individual, and the ability of the substance to elicit a desired response in the individual. Dosing regimen may be adjusted to provide the optimum therapeutic response. For example, several divided doses may be administered daily, or the dose may be proportionally reduced as indicated by the exigencies of the therapeutic situation.

### B. Overview

Kratom products contain multiple pharmacologically active compounds giving the products a complex pharmacology. Traditional use of kratom includes the leaves being chewed to sustain energy or brewing the leaves into a tea or decoction for opioid like analgesia and sedation. It has also been used as a substitution for opium and a preventative for opium withdrawal symptoms⁴. Now, kratom is becoming more commonly used in the US to aid those suffering from opioid use disorder (OUD). A survey of approximately 3000 kratom users indicated that 10% of them used it for OUD with 90% reporting it alleviated their symptoms⁵. However, there are currently no FDA approved indications for mitragynine, so all consumption occurs without medical guidelines.

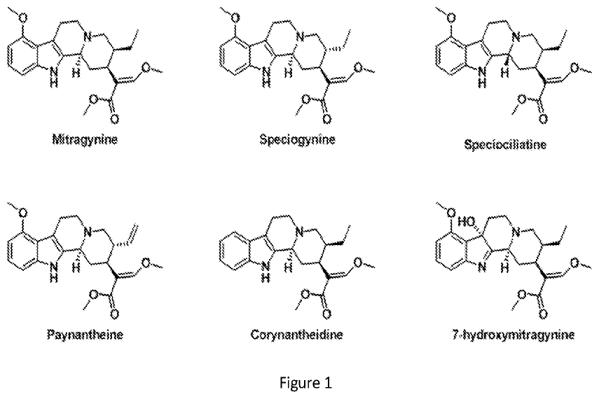

The whole plant material of kratom contains a multitude of biologically active compounds (FIG. 1). Among them, the most abundant alkaloid is mitragynine, while other major indole alkaloids include corynantheidine, speciogynine, speciociliatine, and paynantheine⁷.

Mitragynine is a low affinity, partial μ-opioid receptor (MOR) agonist that differs from standard MOR agonists in that it does not activate the β-arrestin-2 signaling pathway, which is often believed to be linked to the cause of many of the adverse effects of MOR agonists, including respiratory depression and constipation³. Study results have also indicated that mitragynine may produce analgesia through nonopioid mediated pathways⁹⁻¹¹ as its discriminative stimulus and rate-decreasing effects are not completely blocked by the opioid antagonists, naloxone and naltrexone¹². Behavioral assays of lower lip retraction and antinociception demonstrated that mitragynine activity can also be blocked by serotonin antagonists¹³ The antinociceptive effects of mitragynine have also been blocked by the α2 adrenergic antagonist idazoxan, indicating that mitragynine is active at multiple receptor subtypes¹². Due to this activity at both MOR and α2 adrenergic receptors, mitragynine may have the potential to aid both opioid use disorder (OUD) and opioid withdrawal symptoms as well as a painkiller without causing abuse problem.

Therefore, utilizing the antinociceptive effect of mitragynine, new kratom extraction- and mitragynine compositions are provided here for treatment of arthritis. Arthritis, often called osteoarthritis, is one of the most common orthopedic diseases in aged dogs that results in destruction of the joints, chronic pain, and difficulty walking, sitting, or standing. In this disclosure, kratom extract powder comprising 45% mitragynine (w/w) was used to determine if kratom could be useful for relieving pain due to osteoarthritis in dogs.

Due to the insoluble nature of the extract powder (<100 μg/mL, aqueous solubility), a formulation was developed, which increased the amount of mitragynine delivered to the systemic circulation by improving solubility and bioavailability when compared with traditional or commercial products.

The proof-of-concept and tolerability studies were carried out in rats at doses of 8 mg/kg and 2 mg/kg oral, and these doses were well tolerated. A single oral dose of 5 mg/kg mitragynine equivalent was tested and well tolerated in female beagle dogs¹⁹, so an initial starting dose of around 5 mg/kg mitragynine was targeted. But this dose produced adverse effects in 2 out of 3 volunteer dogs which prompted a reformulation. The unexpected adverse effects of kratom extract may be caused by several other alkaloids >5% w/w (speciogynine, speciociliatine, paynantheine) contained in the kratom extract, whose individual safety and tolerability in dogs has not been studied. The increased delivery of these additional minor alkaloids along with mitragynine in the formulation might have increased their bioavailability, and this could account for the unexpected adverse effects.

The clinical study in volunteer dogs using the reformulated capsules had preliminary results indicating that the kratom capsule had a positive effect on pain in osteoarthritic dogs. The placebo-controlled arm of the clinical trial is underway.

### C. Embodiments of the Invention

### Kratom Extracts and Active Alkaloids

Active agents that are considered useful according to the embodiments of the invention include kratom (Mitragyna speciosa) vegetation, including leaves, stems, flowers, and roots thereof, which may be fresh or dried. Kratom leaves are a common source of active alkaloids. Kratom extracts are also considered useful according to the embodiments of the invention. The extracts can include dried and processed (powdered or crushed) kratom vegetation, or prepared as a tea or decoction, or as an alcoholic tincture suitable for oral ingestion. Kratom extracts are commercially available, for example, from Worldseedsupply (worldseedsupply.com/product/mitragyna-speciosa-kratom-45-concentrated-powder-extract-wholesale), Mitrascience (mitrascience.com/product/bulk-kratom-extracts), Overseasorganix (mitrascience.com/product/bulk-kratom-extracts) or Herbal Gifts LLC (thekratomstore.com/product/kratom-45-extract-powder). Any of these commercially available sources of kratom extracts are contemplated for use with the invention.

Preferred kratom extracts contain a known concentration of mitragynine and other alkaloids or are analyzed to determine the concentration of mitragynine. In addition, purified or semi-purified mitragynine or other kratom alkaloids can be used. In alternative embodiments, kratom alkaloids can be combined with other materials such as joint supplements (such as glucosamine chondroitin, fish oils, and green-lipped mussel, for example). Mitragynine is the major alkaloid found in kratom and it as well as other kratom alkaloids and the use of these compounds to aid in opioid use disorder, pain, and other indications are currently being investigated. Due to the insoluble nature of the powdered kratom extract (<100 μg/mL in aqueous solution), herein a formulation was developed to improve solubility and bioavailability of the extract.

### General Pharmaceutics

The active ingredient compound for pharmaceutical composition discussed herein can be present in the form of pharmaceutically acceptable salts, acids, hydrates, and solvates, or as a base. Preferably, the pharmaceutical compositions comprise a therapeutically effective amount of mitragynine (freebase or salts).

In preferred embodiments, the compounds described herein are produced and administered as a pharmaceutical composition that includes kratom extract or one or more kratom alkaloids extracted from the kratom extract as well as one or more pharmaceutically acceptable excipients. A pharmaceutically acceptable excipient refers to any convenient compound or group of compounds that is not toxic and that does not destroy or significantly diminish the pharmacological activity of the therapeutic agent with which it is formulated. Such pharmaceutically acceptable excipients encompass any of the standard pharmaceutically accepted solid, liquid, or gaseous substances known in the art, such as those discussed in the art. The composition described herein can be co-administered with an additional agent, such as a pain medication suitable for the subject which is a drug of another class.

A suitable excipient for a particular pharmaceutical composition depends on the route of administration and the chemistry and pharmacology of the active compounds themselves. Routes of administration are determined by the person of skill according to convenience, the health and condition of the subject to be treated, and the location and stage of the condition to be treated. For example, routes of administration can be local or systemic. Local routes of administration include topical, intra-articular injection, intra-thecal injection, retrobulbar injection; and systemic routes of administration include oral, sublingual/buccal, rectal, cutaneous, inhalation, nasal, parenteral (sub-cutaneous, intra-muscular, intra-venous, intra-dermal), and the like. As for pharmaceutical compositions of the current invention, ER/XR (extended release), SR (sustained release), CR (controlled release), DR (delayed-release), and IR (immediate release) dosage forms can also be contemplated for use with- and in the inventive embodiments. Thus, suitable excipients can be any of the known substances to achieve a desired medication's effect of the active components, herein mitragynine.

Although kratom extract powder or mitragynine can be administered through parenteral route as an injection solution (e.g., 5-10 mg/ml, in particular 8 mg/mL) if need be, preferred embodiments of this invention are to be delivered by the oral route. The forms which the inventive mixture of pharmaceutical composition can take include, but are not limited to: solution, suspension, emulsion, elixir, syrup, paste, tablet (solid, chewable or lozenges, caplet), capsule, granule, powder, premix, and medicated block. In this disclosure, for the purpose of oral therapeutic administration, the active compound is mixed with excipients, especially solubility enhancing excipients and capsule weight filling excipients. The selection of capsule size and capsule fill weight may depend on the body weight of the subject, e.g., gelatin or vegetarian capsules of size 1 (fill weight: 225-500 mg), size 2 (fill weight:165-370 mg), size 3 (fill weight:135-300 mg), size 4 (fill weight: 95-210 mg), and size 5 (fill weight: 60-130 mg). For example, for small size dogs with body weight of less than 15 kg, size 5 or 4 capsule may be used. For medium size dogs of body weight between 15-60 kg, size 3, 2, or 1 capsule may be used. In this disclosure, as a preferred embodiment, the mixtures of 250 mg are packed into a size 1 capsule, targeting a subject of a dog of bodyweight 15-60 kg. However, the mixture can also be formulated into another solid dosage form such as powder, granule or tablets/caplets.

Formulations for a solid dosage form for oral administration generally comprises at least an inert diluent and/or other edible excipients. For example, as diluent, calcium carbonate (CaCO₃), calcium phosphate (Ca₃(PO₄)₂), dibasic calcium phosphate (dicalcium phosphate (CaHPO₄)), tribasic calcium sulfate cellulose, microcrystalline cellulose, dextrates, dextrin, dextrose, fructose, kaolin, lactose, mannitol, sorbitol, starch, pregelatinized starch, sugar, compressible sugar, confectioner's sugar, and the like can be considered. As binder, acacia, alginic acid, carboxymethyl cellulose, sodium cellulose, liquid guar gum, hydroxypropyl methyl cellulose, Povidone-k30, pregelatinized starch syrup, etc. can be considered. In addition to these, glidants for granules, such as calcium silicate, magnesium silicate, silicon dioxide, colloidal talc, and the like, and lubricant for tablets and hard capsules, such as calcium stearate, magnesium stearate, mineral oil, light PEG, purified talc, vegetable oil, stearic acid, and the like can be included. For tablets and capsules, disintegrant to ensure the rapid break down into their primary particles and to facilitate the dissolution or release of the active ingredients, can be included, e.g., alginic acid, cellulose, microcrystalline cellulose, croscarmellose sodium, cross povidone, sodium starch glycolate, pregelatinized starch, and the like. For tablet coating agent, carboxymethyl cellulose, cellulose acetate, cellulose acetate pthalate (CAP), ethyl cellulose hydroxypropyl methylcellulose (HPMC), hydroxypropyl methyl cellulose pthalate, methacrylic acid copolymer, methyl cellulose, PEG, polyvinyl acetate, shellac, titanium dioxide, wax, carnauba wax, microcrystalline zein, etc. can be considered.

The mixture of active ingredient compound and excipients can also be formulated into liquid dosage form for oral administration. Oral liquid dosage form may comprise antioxidants, mineral salts, pH adjusters, preservatives, solvents, stabilizers, surfactants, taste modifiers, thickeners, tonicity adjusters, and the like. In most cases, liquid dosage forms for oral administration can be contained in a container such as a vial or bottle, optionally light protective glass bottle.

In this disclosure, the preferred embodiments of the invention are formulated as mixtures of kratom extract powder comprising 45% mitragynine or mitragynine freebase- or salt form with excipients enhancing solubility, stability and/or permeability (e.g., Captisol®, Kollidon®CL, Kolliphor® P407, and colloidal silicon dioxide) as well as capsule weight filler (e.g., Avicel®). In addition to being packed into capsules or pressed into tablets, which can be individually packed in blister packs or bulk packed in a container, such mixtures can be in the form of a loose powder or granule, divided into doses and packed into individual bags or pouches, or a container in bulk form (e.g., glass or plastic bottle, or metal can) for use as herbal tea or other type of beverage.

### Preferred Kratom Compositions

Preferred compositions of this invention are summarized in Tables I and 2 below. The dosage amounts can be varied by adjusting the amount of microcrystalline cellulose in the formulation, or by administering a smaller or larger amount of the composition.

TABLE 1
Preferred Bulk Kratom Compositions

| | Kratom Extract | Polyanionic Beta | | |
|---|---|---|---|---|
| | Powder (45_% | Cyclodextrin | | Microcrystalline |
| Composition | mitragynine) or | Derivative | Crospovidone | Cellulose |
| Number | equivalent (wt %) | (wt %) | (wt %) | (wt %) |
| 1 | 6.5 | 6.5 | 12 | 75 |
| 2 | 13.0 | 13.0 | 24.0 | 50.0 |
| 3 | 19.5 | 19.5 | 36.0 | 25.0 |
| 4 | 24.7 | 24.7 | 45.6 | 5.0 |
| 5 | 26 | 26 | 48 | 0 |

| | Kratom extract | Captisol ® | | Avicel ® | Total |
|---|---|---|---|---|---|
| | powder (45% | (Polyanionic beta | Kollidon ®CL | Microcrystalline | capsule |
| | mitragynine) | cyclo-dextrin | (cros-povidone) | Cellulose | fill weight |
| Ex. No. | (mg) | derivative) (mg) | (mg) | (mg) | (mg) |
| 6 | 20 | 20 | 40 | 170 | 250 |
| 7 | 20 | 25 | 40 | 165 | 250 |
| 8 | 20 | 30 | 40 | 160 | 250 |

TABLE 2
Preferred Kratom Dosage Forms

| | Total Kratom | | | | |
|---|---|---|---|---|---|
| | Extract Powder | | | Subject | |
| Dosage | (w/45% | Calculated | Mitragynine | Body | |
| Unit | mitragynine) | mitragynine | Dose | Weight | Capsule |
| Number | (mg) | (mg) | (mg/kg) | (kg) | Size |
| 1 | 20 | 9 | 0.5 | 18 | 1 |
| 2 | 40 | 18 | 0.5 | 36 | 1 |
| 3 | 60 | 27 | 0.5 | 54 | 1 |

Preferred kratom formulations contain about 4% to about 30% kratom extract powder, or an equivalent amount to contain about 2% to about 10% mitragynine to the composition. The most preferred formulations contain about 8% to about 10% kratom extract powder, or an equivalent amount to contain about 4% to about 5%.

Preferred kratom extract powder formulations contain about 2% to about 40% polyanionic beta cyclodextrin derivative, optionally about 4% to 16%. The most preferred formulations contain about 8% to about 12% polyanionic beta cyclodextrin derivative. A preferred polyanionic beta cyclodextrin derivative is CAPTISOL@, but sulfobutyl ether (SBE) beta cyclodextrin can also be used as a substitute in some embodiments. The ratio (w/w) of kratom extract powder vs. polyanionic beta cyclodextrin derivative can be 1:0.5 to 1:2, optionally 1:1 to 1:1.5.

Preferred kratom extract powder formulations contain about 30% to about 50% crospovidone. The most preferred formulations contain about 40% crospovidone. A preferred crospovidone is KOLLIDON® CL. Crospovidone XL crosslinked polyvinylpyrrolidone, or other crospovidone can be used as a substitute in some embodiments.

The remaining capsule weight is filled with microcrystalline cellulose. A preferred microcrystalline cellulose is Avicel® or Avicel® PH-101. Comprecel® or generic microcrystalline cellulose can be used as a substitute in some embodiments.

### Doses and Regimens

Dosage amounts per administration include any amount determined by the practitioner, and will depend on the size and weight of the subject to be treated, the state of the health of the subject, the severity of the condition to be treated, and the like. In general, it is contemplated that for the majority of subjects, for example dogs, a dose in the range of about 0.2 mg/kg to about 1 mg/kg is suitable, preferably about 0.3 mg/kg to about 0.9 mg/kg, more preferably about 0.4 mg/kg to about 0.8 mg/kg, and most preferably about 0.5 mg/kg to about 0.7 mg/kg are useful. This dose can be administered, for example, once a day, twice a day at dosing interval of 12 hours, or thrice a day at dosing interval of 8 hours. For example, daily doses can be about 5 mg once a day or about 30 mg thrice a day, depending on the body weight of a subject and severity of the condition.

Dosing schedule for treatment regimens can be a single administration or a course of administrations lasting two or more days, including a week, two weeks, several weeks, a month, two months, several months, a year, or more, including administration for the remainder of the subject's life.

### Subjects

Preferred subjects for use with embodiments of the invention are canines, but subjects also can include simians, humans, avians, felines, equines, rodents, bovines, porcines, ovines, caprines, lagomorphs and other exotic species. Subjects in need include any subject that has a painful condition such as osteoarthritis, is suspected of having a painful condition such as osteoarthritis, is susceptible to having a painful condition such as osteoarthritis, and the like. Osteoarthritis is typically associated with chronic pain, but other painful conditions for a subject in need include acute pain, neuropathic pain, nociceptive pain, radicular pain, psychogenic pain etc. or other painful conditions.

### Methods of Treatment

The invention also relates to methods of treatment for pain conditions in a subject in need, preferably in a canine with osteoarthritis. For treatment of osteoarthritis in canines, a dog is administered a dose of the compositions according to the invention, preferably as a capsule dosage form. Appropriate doses are based on the weight of the subject and are discussed above. In summary, a canine subject preferably is administered at a dose of about 0.5 mg/kg to about 0.7 mg/kg or about 15 mg to about 20 mg for a subject weighing about 30 kg. This dosage preferably is given two to three times a day for a period of 7-28 days. This dose may be administered on an “as needed” basis or administered as a regimen. Escalation to the target dose range over a couple of days to weeks may be instituted. Escalation to desired effect within the target dose range may be instituted. This dose may be started or stopped without de-escalation. In addition, the composition can be given about 1 hour before surgery, and after the surgery, similar regimens can be applied to the subject.

### D. Summary of Results

A formulation was developed that increased the amount of mitragynine delivered to the systemic circulation compared with traditional or commercial products. Proof-of-concept and tolerability studies were carried out in rats at doses of 8 mg/kg and 2 mg/kg, and these doses were well tolerated. Although a single oral dose of 5 mg/kg mitragynine was well tolerated in female beagle dogs, this dose produced adverse effects in 2 out of 3 volunteer dogs in an initial study, which prompted a change to the dose and formulation. The inventive formulation therefore contained 10 mg of mitragynine. This formulation achieves the desired systemic exposure of mitragynine at half the dose and is suitable for development as a pharmaceutical for human use.

### E. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1. Materials and Methods

### 1.1 Chemicals and Reagents

Captisol® (Cas No. 182410-00-0) was generously donated by Captisol (CyDex Pharmaceuticals Inc, Lenexa, KS, USA). Kollidon® CL (CAS No. 9003-39-8) and Kolliphor P407 Geismar (P407) (CAS No. 9003-11-6) were generously donated by BASF (Florham Park, NJ, USA). Avicel® PH101 (CAS No. 9004-34-6) and verapamil hydrochloride [(purity >98%); internal standard (IS)] were purchased from Sigma (St. Louis, MO, USA). Colloidal silicon dioxide (CSD) (CAS No. 7631-86-9) was purchased from Spectrum Chemical (New Brunswick, NJ, USA)

Liquid chromatography-mass spectrometry (LC-MS) grade acetonitrile, methanol, water, and formic acid, and size 9 gelatin capsules (Torpac, Fairfield, NJ, USA) were purchased from Fisher Scientific (Hampton, NH, USA). Size 1 vegetable capsules were sourced from Xprs Nutra (South Jordan, UT, USA). Ethanol, hexanes, ethyl acetate, methylene chloride, hydrogen chloride, diethyl ether, methanol, acetonitrile, and sulfuric acid solution were purchased from Acros Organic (Carlsbad, CA, USA).

Kratom powder extract (45% mitragynine w/w) was obtained from Choice Organics. Individual kratom alkaloids mitragynine, corynantheidine, speciogynine, speciociliatine, paynantheine, and corynoxine were isolated and purified from a kratom alkaloid rich extract as previously described⁷. The 7-hydroxymitragynine was semi-synthetically obtained from mitragynine. All the alkaloids were >99% pure, with the structure and purity confirmed by proton (¹H) nuclear magnetic resonance spectroscopy (NMR), carbon (¹³C) NMR, ultra-high-performance liquid chromatography photodiode array detection (UHPLC-PDA), and liquid chromatography high-resolution quadrupole time-of-flight mass spectrometry (LC-Q-TOF).

### 1.2 Methods for Kratom Extract Formulation

1.2.1 Formulation Development and In Vitro Dissolution Study with Kratom Extract Powder

Captisol® is a polyanionic beta cyclodextrin derivative with a sodium sulphonate salt separated from the lipophilic cavity by a butyl ether spacer group. This powder was used here as a solubility and permeability enhancer for kratom alkaloid compounds. It has been found to be safe for use in dogs up to 1900 mg/kg/day. Kollidon® compounds are povidones, copovidones, and crospovidones, and they are versatile materials that are most commonly used for solid oral dosage forms. Kollidon® CL is a crosslinked water insoluble polyvinyl pyrrolidone (crospovidone) supplied as a fine, white to almost white powder that is used as a super disintegrant and dissolution enhancer. The oral LD₅₀ is >2000 mg/kg in rats and 2500 mg/kg/day in beagle dogs. Other sources of crospovidone are known in the art and can be used in the inventive formulations. Avicel® (CAS No. 9004-34-6) is microcrystalline cellulose supplied as a fine, white powder used as a filler with an oral LD₅₀ of >5000 mg/kg in rats. AVICEL® PH-101 has a particle size of about 50 μm.

Formulations of different ratios of kratom extract powder to Captisol® were used in the dissolution study with the remaining weight of the capsule filled with Kollidon® CL and Avicel®. Ratios of 1 to 0.5, 1 to 1, 1 to 1.5, and 1 to 2 of kratom extract to Captisol® were investigated to determine which had the best solubility properties at pH 1.2.

A USP Type 11 apparatus was used to carry out all dissolution studies. The paddle attachment was used with 750 mL of buffered water (pH adjusted to 1.2 with 10 M hydrochloric acid). Paddle speed was set to 75 rpm and buffer temperature was held constant at 37.0±0.5° C.

Samples (2 mL) were drawn at 0, 15, 30, 45, and 60 min and filtered with a 0.2 μm syringe filter.

1.2.2 Animals for a Proof of Concept and Safety Study with Kratom Extract

### 1.2.2.1 Rat Study

Once the capsule formulation was finalized, a proof of concept and safety study was carried out in Sprague Dawley rats. Male and female rats (250±25 g) were purchased from Envigo (Indianapolis, IN, USA) with indwelling jugular vein cannulas and acclimated ≥72 h in a temperature- and humidity-controlled vivarium on a 12 h light/dark cycle. Study protocols were approved by the Institutional Animal Care and Use Committee (IACUC) at the University of Florida, which is fully accredited by the Association for Assessment and Accreditation of Laboratory Animal Care International (AAALAC) and were written in accordance with the National Institutes of Health (NIH) Guide for the Care and Use of Laboratory Animals.

Rats were fasted for 10 h prior to dosing and an additional 2 h post-dose. Rats were dosed with one size 9 gelatin capsule with fill weights of 19.5±0.5 mg, (about 5.1 mg kratom or 2.3 mg mitragynine). After capsule dosing, 0.5 mL of water was provided by oral gavage to ensure capsule did not lodge in the esophagus. Rats were connected to an automatic blood collection system (Bioanalytical Systems Inc., West Lafayette, IN, USA) and blood samples were taken at pre-dose, 0.08, 0.17, 0.25, 0.5, 0.75, 1, 2, 4, 6, 8, 12, 18, and 24 h. At the completion of the study, plasma samples were separated by centrifugation at 850×g for 10 min at 4° C. and stored at −20° C. until analysis.

### 1.2.2.2 Dog Study:

Client owned dogs (weight range 25-45 kg) of any breed or sex, presenting with naturally occurring osteoarthritis were used. The study design was a randomized, double-blinded, placebo-controlled, crossover clinical trial. Dogs were required to have a 14-day washout period for pain medications such as gabapentin and tramadol and/or a 30-day washout period for glucocorticoids prior to enrollment. Dogs would receive kratom capsules every 8 hours for 4 weeks with a 2-week washout period in between followed by 4 weeks of the opposite treatment. Initial tolerability study in dogs were performed on N=3 to determine the appropriate dose range.

1.2.3 Analysis of the Proof of Concept Study in Rats with Kratom Extract Formulation

Primary stock solutions of all alkaloids were prepared by weighing and dissolving an appropriate amount in acetonitrile to get 1 mg/mL stocks. These primary stocks were then further diluted to get secondary stocks of 10 μg/mL and 1 μg/mL of each alkaloid. The mix stocks were used to prepare mix stocks at concentrations of 25, 50, 250, 500, 1000, 1500, 2500, and 5000 ng/mL. Quality control standards (QC) stocks were made from a different set of primary stocks at 25, 62.5, 2000, and 4000 ng/mL.

The 45% mitragynine (w/w) kratom extract powder was analyzed for content by dissolving 10 mg of the powder in 30 mL acetonitrile. The solution was then diluted to fit into the calibration range (2.5-500 ng/mL) with methanol containing 0.05% (v/v) formic acid and 50 ng/mL verapamil (IS). Dissolution study samples were diluted to fit within the linear range with methanol containing 0.05% (v/v) formic acid and 50 ng/mL IS prior to analysis. The powder extract was also analyzed for pesticides, heavy metals, microbial and/or mycotoxin contamination, residual solvents, and filth.

To process plasma (rat or dog) samples for analysis, 20 μL aliquots of each sample were taken. To prepare calibration standards (CS), 2.0 μL of mix stock were added to 18 μL of blank plasma. This generated eight standards of 2.5, 5, 25, 50, 100, 150, 250, and 500 ng/mL, in the respective plasma. The same process was used to generate the lower limit of quantification (LLOQ) (2.5 ng/mL), low quality control (LQC) (6.25 ng/mL), medium quality control MQC (200 ng/mL) and high quality control (HQC) (400 ng/mL). After spiking, the CS and QC samples were vortex mixed for 5 min at 650 rpm. Analytes were precipitated using 100 μL methanol acidified with 0.05% formic acid containing 50 ng/mL IS in a 1:4 ratio. The samples were then vortex mixed for 5 min at 650 rpm before being transferred to a 96-well Millipore Solvinert 0.45 μm filter plate (Burlington, MA, USA) and centrifuged at 850×g for 2 min at 4° C.

A previously reported UPLC-MS/MS method for the quantification of eleven kratom alkaloids was used to analyze all samples¹³. Briefly, the mobile phase composed of aqueous ammonium acetate buffer (2.5 mM, pH 3.5, (A)) and acetonitrile (B). The gradient started at 90% A and 10% B, and A was decreased to 75% by 3.5 min and then to 60% by 10 min. At 10 min, there was a sharp increase of B to 70% by 10.2 min, which was then dropped to 10% by 10.5 min, where it was maintained until 11 min. An Acquity UPLC® BEH C18 column (1.7 μm, 2.1×100 mm) with a VanGuard precolumn of the same chemistry was used for separation (Waters, Milford, MA, USA). The UPLC-MS/MS was a Waters™ Acquity Class-I chromatograph coupled with a Xevo TQ-S Micro triple quadrupole mass spectrometer (Waters, Milford, MA, USA).

The TargetLynx™ application of MassLynx™ 4.2 was used for data processing and quantification of the UPLC-MS/MS data (Waters, Milford, MA, USA). Phoenix Version 6.4 (Certara, Princeton, NJ, USA) was used for the non-compartmental analysis using a linear log interpolation. Graphpad Prism Version 8 (GraphPad Software, San Diego, CA, USA) was used to plot the concentration-time profiles for each dataset. R Studio version 1.4.1717 was used for statistical analysis (R Foundation for Statistical Computing, Vienna, Austria).

### Example 2. Characterization of Kratom 45% Extract

The results of the quantification of the 45% mitragynine (w/w) kratom extract powder showed mitragynine was present as 44.1% w/w followed by paynantheine, speciociliatine, speciogynine, and corynantheidine (Table 3).

TABLE 3
Analysis of alkaloid content in 45% mitragynine (w/w) extract powder

| Alkaloid | Mitragynine | Corynantheidine | Paynantheine | Speciogynine | Speciociliatine |
|---|---|---|---|---|---|
| % w/w | 44.1 | 1.2 | 8.6 | 4.6 | 4.9 |

The 45% mitragynine (w/w) kratom extract powder passed testing for filth, residual solvents, heavy metals, mycotoxins, pesticides, microbial, and total yeast and mold (Table 4). Only residual solvents and pesticides that are above the detection limit are included in the table.

TABLE 4
Raw material testing of 45% mitragynine (w/w) kratom extract powder

| | | | Unit of | Action | |
|---|---|---|---|---|---|
| Test | Analyte | Result | Measure | Limit | Pass/Fail |
| Solvents | Dichloromethane | 336.18 | ppm | 600 | Pass |
| Solvents | Ethyl acetate | 58.34 | ppm | 5000 | Pass |
| Solvents | Chloroform | 19.721 | ppm | 60 | Pass |
| Solvents | Toluene | 37.341 | ppm | 890 | Pass |
| Microbial | Escherichia | Not present | CFU | 1726 | Pass |
| | coli/Shigella spp. | in 1 gram. | | | |
| | Salmonella | | | | |
| Microbial | Aspergillus flavus, | Not present | CFU | 10,000 | Pass |
| | Aspergillus | in 1 gram. | | | |
| | fumigatus, | | | | |
| | Aspergillus niger, | | | | |
| | Aspergillus terreus | | | | |
| Mycotoxins | Aflatoxin G2, G1, B2, | ND | ppm | 0.02 | Pass |
| | B1, Ochratoxin A+ | | | | |
| Mycotoxins | Total Mycotoxins | ND | ppm | | Pass |
| Heavy metals | Arsenic | ND | ppm | 1.5 | Pass |
| Heavy metals | Cadmiuim | ND | ppm | 0.5 | Pass |
| Heavy metals | Mercury | ND | ppm | 3 | Pass |
| Heavy metals | Lead | 0.273 | ppm | 0.5 | Pass |
| Pesticide | Thiamethoxam | 0.072 | ppm | 1 | Pass |
| Filth/foreign | Filth and Foreign | ND | Detect/g | 3 | Pass |
| material | Material | | | | |
| Total yeast | Total Yeast and Mold | <10 | CFU | 100,000 | Pass |
| and Mold | | | | | |
| Total yeast | Quantitative Total EC | ND | CFU/g | | Pass |
| and Mold | | | | | |
| Total yeast | Quantitative BTGN | ND | CFU/g | | Pass |
| and Mold, | | | | | |
| Total yeast | Quantitative Total | ND | CFU/g | | Pass |
| and Mold | Aerobic Bacteria | | | | |

ppm = parts per million;
CFU = colony forming units;
ND = not detected
BTGN = bile-tolerant gram-negative bacteria

### Example 3. Dissolution Study with Kratom Extract Formulation

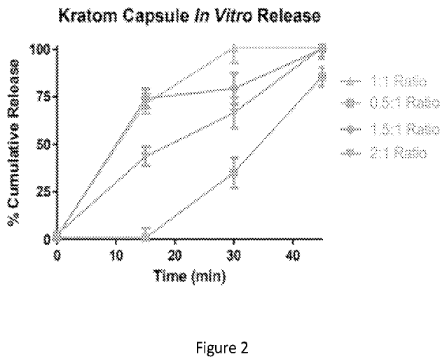

FIG. 2 shows the in vitro release profile of 4 different ratios of Captisol® to kratom extract powder. The ratio of 1 to 1 of Captisol® to 45% mitragynine (w/w) kratom extract powder showed the best release profile (>80% after 30-min), so it was selected as the ratio for the final formulation.

The ratio of 1.5 to 1 of Captisol® to 45% mitragynine (w/w) kratom extract powder had the second-best release profile of ˜75% by 15 min while the 0.5 to 1 and 2 to 1 formulations did not reach 80% release until >30-min.

### Example 4. Bioavailability Study in Rats

Pharmacokinetic parameters following a single capsule dose in male Sprague Dawley rats can be seen in Table 5. Compared with a traditional preparation (lyophilized kratom tea solution) and a commercially available kratom product (OPMS Gold liquid kratom extract), the bioavailability of mitragynine increased 45 and 29%, respectively with the inventive capsule formulation. The Cₘₐₓ, of mitragynine was increased 4-fold when delivered by capsule as compared with the two oral solutions. The exposure was also greatly increased by 18- and 3-fold as compared with the LKT and OPMS solutions, respectively.

These results show superiority of the inventive formulation (2-fold increase in maximum concentration, systemic exposure, and bioavailability of mitragynine compared to an oral solution of mitragynine; 4-fold increase in maximum concentration compared to LKT and commercial product; 3-fold and 2-fold increase in systemic exposure compared to LKT and commercial product, respectively) in delivering mitragynine systemically.

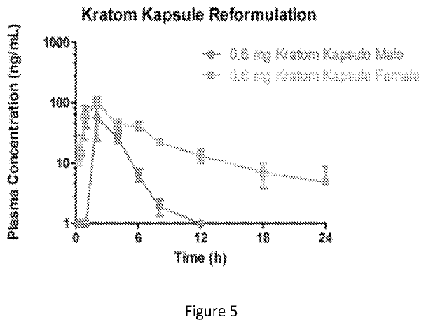

The proof-of-concept study demonstrated that the capsule formulation of the invention was able to effectively deliver mitragynine to the systemic circulation. The mean plasma concentration-time profiles of the three different kratom preparations can be seen in FIG. 5.

TABLE 5
Pharmacokinetic parameters in male rats following oral dose of a lyophilized kratom
tea (LKT) solution, a commercially available OPMS Gold liquid solution, or a 45%
mitragynine (w/w) kratom extract capsule. Values represent the mean (N = 4) ± SEM

| | | Mitragynine | LKT | OPMS | Inventive |
|---|---|---|---|---|---|
| | | solution | solution | solution | formulation |
| | _Mitragynine_{——}_{——}_{——} | oral | oral | oral | oral |
| Parameter | IV (2.5 mg/kg) | (20 mg/kg) | (5.7 mg/kg) | (9.6 mg/kg) | (8 mg/kg) |
| Cₘₐₓ/Dose | — | 22.7 ± 4.9 | 11.1 ± 1.1 | 11.7 ± 1.6 | 46.1 ± 9.5 |
| (μg/L/mg/kg) | | | | | |
| Tmax (h) | — | 0.4 ± 0.1 | 1.3 ± 0.3 | 3.1 ± 0.4 | 1.3 ± 0.3 |
| AUC/Dose | 335 ± 16.5 | 122.4 ± 13.0 | 83.7 ± 6.4 | 136.1 ± 13.1 | 232.9 ± 65.9 |
| (μg*hr/L/mg/kg) | | | | | |
| Bioavailability | | 37 | 25 | 41 | 69 |
| (%) | | | | | |

Cₘₐₓ/Dose = maximum concentration adjusted for dose;
Tₘₐₓ = time to reach Cₘₐₓ;
AUC_{0-∞}/dose = area under the concentration-time curve from time 0 to infinity (exposure) adjusted for dose;
F = absolute oral bioavailability

### Example 5. Clinical Study in Osteoarthritic Dogs

A tolerability study was carried out in 3 volunteer dogs. The capsules contained 40 mg of mitragynine per capsule. The first dog received a dose of 3-4 mg/kg mitragynine but showed signs of dysphoria, whining, restlessness, accidents in the house, and would not eat. Due to this, the owner removed the dog from the study. A second dog was given a dose of 2 mg/kg but also showed signs of whining, restlessness, and had a lack of interest in food. A third dog was given a dose of 1 mg/kg which was tolerated. Because two out of three dogs had a reaction to the capsule containing 40 mg of mitragynine, a reformulation was necessary.

To determine the target dose, a survey was conducted on dog owners who supply kratom to their dogs from commercial sources. The owners reported the amount of kratom they dosed to each dog and from this information the approximate mitragynine equivalent dose was calculated (Table 6). Over 70% of owners reported that they use kratom to help with pain relief and over 70% reported no adverse effects. Based on the results of the survey, a capsule containing 10 mg mitragynine (4-fold reduction) was determined to be appropriate as it would result in a starting dose of approximately 0.3 mg/kg.

TABLE 6
Survey results for clients giving commercial
kratom products to their dogs.

| | Total | | Amount of | Delivered |
|---|---|---|---|---|
| | Kratom | Dog | Mitragynine | Mitragynine |
| | Dose | Weight | per dose* | dose** |
| Subject | (mg) | (kg) | (mg) | (mg/kg) |
| 1 | 500 | 23 | 10 | 0.21 |
| 2 | 1250 | 6.8 | 25 | 1.83 |
| 3 | 625 | 22 | 12.5 | 0.28 |
| 4 | 313 | 36 | 6.26 | 0.08 |
| 5 | 2000 | 30 | 40 | 0.66 |
| 6 | 250 | 2.7 | 5 | 0.92 |
| 7 | 1000 | 30 | 20 | 0.33 |
| 8 | 625 | 27 | 12.5 | 0.23 |
| 9 | 313 | 6.8 | 6.26 | 0.46 |
| 10 | 313 | 5.5 | 6.26 | 0.56 |
| 11 | 625 | 13.6 | 12.6 | 0.45 |
| 12 | 1000 | 34 | 20 | 0.29 |
| 13 | 2500 | 6.8 | 50 | 3.67 |
| 14 | 2500 | 18 | 50 | 1.38 |

*assuming 2% of dried leaf material (w/w) is mitragynine;
**assuming 50% bioavailability.

### Example 6. Reformulation of 45% Mitragynine (w/w) Kratom Extract Capsule

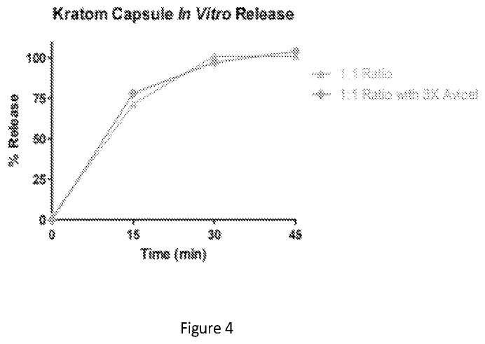

To dilute the powder formulation from a capsule containing 40 mg of mitragynine to a capsule containing 10 mg of mitragynine, Avicel® was added at a 1 to 3 ratio of the original formulation to Avicel®. A dissolution study was performed to ensure that the in vitro release profile of mitragynine was unchanged. There was no change in the dissolution profile with the addition of 3X Avicel (FIG. 4).

Additionally, a proof-of-concept and tolerability study was performed in 4 male and 4 female rats to confirm the 4-fold dose reduction resulted in a 4-fold decrease in Cₘₐₓ and AUC. The results of the study on the reformulation can be seen in Table 7 and FIG. 5. The Cₘₐₓ in males was decreased by 4-fold while the AUC was decreased 6-fold.

TABLE 7
Reformulation proof of concept pharmacokinetic results in rats.
Values represent the mean (N = 4) ± SEM.

| | Kratom | Kratom | Kratom |
|---|---|---|---|
| | Capsule | Capsule | Capsule |
| | High Dose | Low Dose | Low Dose |
| | (8 mg/kg) | (2 mg/kg) | (2 mg/kg) |
| Parameter | Male | Male | Female |
| Cₘₐₓ/Dose | 46.1 ± 4.8 | 55.8 ± 14.1 | 60.8 ± 5.9 |
| (ng/mL/mg/kg) | | | |
| Tₘₐₓ (h) | 1.3 ± 0.3 | 2.0 ± 0.0 | 1.7 ± 0.3 |
| AUC/Dose | 232.9 ± 33.0 | 128.6 ± 5.4 | 286.1 ± 8.1 |
| (ng*hr/mL/mg/kg) | | | |
| F (%) | 69.4 | 38.3 | 56.6 |

Cₘₐₓ/Dose = dose normalized maximum plasma concentration;
Tₘₐₓ = time to reach Cₘₐₓ;
AUC/Dose = dose normalized area under the concentration-time curve (exposure);
F = absolute oral bioavailability

### Example 7. Osteoarthritic Dog Study Results

Using the 45% mitragynine (w/w) kratom extract, a dose-escalation pilot study using 3 dogs was performed to evaluate the utility and safety of kratom in the management of naturally occurring osteoarthritis of the carpus, elbow, shoulder, tarsus, stifle, and/or hip joint, and to identify ideal dosing range and frequency to achieve an analgesic response. Dogs were treated with 0.32-0.35 mg/kg orally every 12 hours with weekly escalations up to 0.5-0.6 mg/kg orally every 8 hours.

Real-time pain levels were acquired during orthopedic exams, using skin-mounted sensors that process a direct pain biosignal generated by the nervous system (PainTrace®, Charlottetown, Prince Edward Island). Additionally, gait analysis was performed using a pressure-sensitive walkway (Tekscan, Boston, MA). Dogs were also fitted with a smart health monitoring collar (PetPace®, Burlington, MA) that continuously records vitals, activity levels, and body positions. Finally, owners were asked to rate the severity of their dog's pain and the degree to which that pain interferes with function using the Canine Brief Pain Inventory (CBPI). Preliminary results reveal that pain scores were overall improved across all assessment types. There was an overall improvement in summative pain noted on the PainTrace®, improvement in symmetry indices on gait analysis, and a dose dependent improvement in function noted on the CBPI.

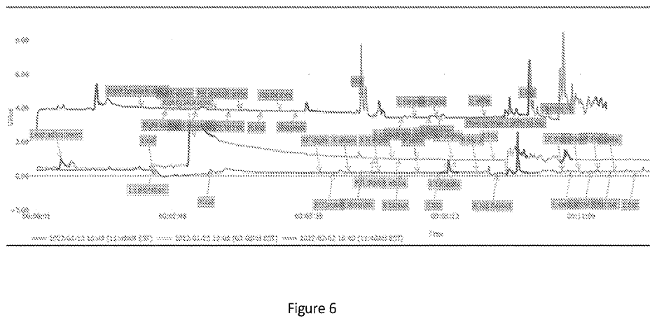

Preliminary results reveal that pain scores were overall improved across all assessment types. There was an overall improvement in summative pain noted on the PainTrace®, improvement in symmetry indices on gait analysis, and a dose dependent improvement in function noted on the CBPI. FIG. 6 shows PainTrace® of Dog 1 after dose and frequency escalation, demonstrating a decline in baseline pain measures (Baseline Delta −0.66>−0.3>−0.02>0.11), denoting summative pain during exam improved.

No adverse effects were noted at low and moderate doses (0.32-0.35 mg/kg every 8-12 hours and 0.5-0.7 mg/kg every 8 hours respectively). Higher doses (3-4 mg/kg orally every 8 hours) resulted in vocalization, and hyporexia. Discontinuation of the drug resulted in resolution of these signs. (FIG. 6)

### REFERENCES

All references listed below and throughout the specification are hereby incorporated by reference in their entirety.
1. Sharma, Abhisheak. and Christopher R. McCurdy. “Assessing the therapeutic potential and toxicity of Mitragyna speciosa in opioid use disorder.” Expert Opinion on Drug Metabolism & Toxicology 17.3 (2021): 255-257.
2, Kamble, Shyam H., et al. “Pharmacokinetics of Eleven Kratom Alkaloids Following an Oral Dose of Either Traditional or Commercial Kratom Products in Rats.” Journal of Natural Products 84.4 (2021): 1104-1112.
3. Eastlack, S. C.; Cornett, E. M.; Kaye, A. D., kratom-pharmacology, clinical implications, and outlook: a comprehensive review. Pain Ther 2020, 9 (1), 55-69.
4. Vicknasingam, B.; Narayanan, S.; Beng, G. T.; Mansor, S. M., The informal use of ketum (Mitragyna speciosa) for opioid withdrawal in the northern states of peninsular Malaysia and implications for drug substitution therapy. Int J Drug Policy 2010, 21 (4), 283-288.
5. Coe, M. A.; Pillitteri, J. L.; Sembower, M. A.; Gerlach, K. K.; Henningfield, J. E., Kratom as a substitute for opioids: Results from an online survey. Drug Alcohol Depend 2019, 202, 24-32.
6. Zhang, M.; Sharma, A.; Ledn, F.; Avery, B.; Kjelgren, R.; McCurdy, C. R.; Pearson, B. J., Plant growth and phytoactive alkaloid synthesis in kratom [Mitragyna speciosa (Korth.)] in response to varying radiance. PLOS ONE 2022, 17 (4), e0259326.
7. Sharma, A.; Kamble, S. H.; Leon, F.; Chear, N. J.; King, T. I.; Berthold, E. C.; Ramanathan, S.; McCurdy, C. R.; Avery, B. A., Simultaneous quantification of ten key Kratom alkaloids in Mitragyna speciosa leaf extracts and commercial products by ultraperformance liquid chromatography-tandem mass spectrometry. Drug Test Anal 2019, 11 (8), 1162-1171.
8. Eastlack, S. C.; Cornett, E. M.; Kaye, A. D., kratom-pharmacology, clinical implications, and outlook: a comprehensive review. Pain Ther 2020, 9 (1), 55-69.
9. Hiranita, T.; Leon, F.; Felix, J. S.; Restrepo, L. F.; Reeves, M. E.; Pennington, A. E.; Obeng, S.; Avery, B. A.; McCurdy, C. R.; McMahon, L. R.; Wilkerson, J. L., The effects of mitragynine and morphine on schedule-controlled responding and antinociception in rats. Psychopharmacol 2019, 236 (9), 2725-2734
10. Matsumoto, K.; Mizowaki, M.; Suchitra, T.; Murakami, Y.; Takayama, H.; Sakai, S.; Aimi, N.; Watanabe, H., Central antinociceptive effects of mitragynine in mice: contribution of descending noradrenergic and serotonergic systems. Eur J Pharmacol 1996, 317 (1), 75-81.
11. Obeng, S.; Kamble, S. H.; Reeves, M. E.; Restrepo, L. F.; Patel, A.; Behnke, M.; Chear, N. J.; Ramanathan, S.; Sharma, A.; Leon, F.; Hiranita, T.; Avery, B. A.; McMahon, L. R.; McCurdy, C. R., Investigation of the adrenergic and opioid binding affinities, metabolic stability, plasma protein binding properties, and functional effects of selected indole-based kratom alkaloids. J Med Chem 2020, 63 (1), 433-439.
12. Reeve, M. E.; Obeng, S.; Oyola, F. L.; Behnke, M.; Restrepo, L. F.; Patel, A.; Ho, N. P.; Williamson, M. R.; Gamez Jimenez, L. R.; McCurdy, C. R.; McMahon, L. R.; Hiranita, T., The Adrenergic a2 Receptor-Mediated Discriminative-Stimulus Effects of Mitragynine, the Primary Alkaloid in Kratom (Mitragyna Speciosa) in Rats. FASEB J 2020, 34 (S1), 1-1.
13. Leon, F.; Obeng, S.; Mottinelli, M.; Chen, Y.; King, T. I.; Berthold, E. C.; Kamble, S. H.; Restrepo, L. F.; Patel, A.; Gamez-Jimenez, L. R.; Lopera-Londono, C.; Hiranita, T.; Sharma, A.; Hampson, A. J.; Canal, C. E.; McMahon, L. R.; McCurdy, C. R., Activity of Mitragyna speciosa (“Kratom”) Alkaloids at Serotonin Receptors. J Med Chem 2021, 64 (18), 13510-13523.
14. Ramanathan, S.; Parthasarathy, S.; Murugaiyah, V.; Magosso, E.; Tan, S. C.; Mansor, S. M., Understanding the physicochemical properties of mitragynine, a principal alkaloid of Mitragyna speciosa, for preclinical evaluation. Molecules 2015, 20 (3), 4915-27.
15. McConnell, E. L.; Basit, A. W.; Murdan, S., Measurements of rat and mouse gastrointestinal pH, fluid and lymphoid tissue, and implications for in-vivo experiments. J Pharm Pharmacol 2008, 60 (1), 63-70.
16. Kamble, S. H.; Berthold, E. C.; King, T. I.; Raju Kanumuri, S. R.; Popa, R.; Herting, J. R.; Leon, F.; Sharma, A.; McMahon, L. R.; Avery, B. A.; McCurdy, C. R., Pharmacokinetics of eleven kratom alkaloids following an oral dose of either traditional or commercial kratom products in rats. J Nat Prod 2021, 84 (4), 1104-1112.
17. Food and Drug Administration (FDA), Bioequivalence Studies With Pharmacokinetic Endpoints for Drugs Submitted Under an ANDA Guidance for Industry. Silver Spring, MD, 2021.
18. Nair, A. B.; Jacob, S., A simple practice guide for dose conversion between animals and human. J Basic Clin Physiol Pharmacol 2016, 7 (2), 27-31.
19. Maxwell E A, King T I, Kamble S H, Raju K S R, Berthold E C, León F, Avery B A, McMahon L R, McCurdy C R, Sharma A. Pharmacokinetics and Safety of Mitragynine in Beagle Dogs. Planta Med. 2020 November; 86(17):1278-1285. doi: 10.1055/a-1212-5475. Epub 2020 Jul. 21.

What is claimed is:

1. A kratom composition comprising:
(a) about 4% to about 30% by weight kratom extract powder comprising about 2% to about 65% mitragynine, or freebase or salt thereof, (w/w), optionally about 8% by weight kratom extract powder comprising about 45% mitragynine (w/w); or about 2% to about 10% by weight mitragynine, optionally 4% by weight mitragynine;
(b) about 2% to about 40% by weight polyanionic beta cyclodextrin derivative, optionally about 4%-16% by weight polyanionic beta cyclodextrin derivative;
(c) about 10% to about 50% by weight crospovidone, optionally about 40% by weight crospovidone; and
(d) the remaining weight is filled with an excipient, wherein the excipient is, optionally, microcrystalline cellulose;
wherein the composition is for oral administration.

2. The kratom composition of claim 1, wherein the composition is in a dosage form of a gelatin- or vegetarian capsule.

3. The kratom composition of claim 2, wherein the size of the capsule can be 1, 2, 3, 4, or 5, in particular size 1.

4. The kratom composition of claim 2, 3, or 4, wherein a total weight in the capsule ranges from 100 mg to 500 mg, in particular 250 mg.

5. The kratom composition of claim 1, wherein the ratio of the kratom extract powder to polyanionic beta cyclodextrin derivative ranges from 1:0.5 to 1:2, in particular 1:0.75 to 1:1.75, and in more particular 1:1 to 1:5.

6. The kratom composition of claims 1 to 5, wherein the composition comprises the kratom extract powder of about 10 mg to 40 mg, in particular about 20 mg to 40 mg; or
mitragynine of about 5 mg to 20 mg, in particular about 10 mg to 20 mg.

7. The kratom composition of claim 1, wherein the composition may comprise further excipients.

8. A mitragynine composition comprising mitragynine and polyanionic beta cyclodextrin derivative for oral administration, wherein the ratio of mitragynine and polyanionic beta cyclodextrin derivative (w/w) is about 1:1 to 1:4, in particular 1:1.5 to 1:35, and in more particular 1:2 to 1:3.

9. The mitragynine composition of claim 8, wherein the total weight of the composition is 100 mg to 500 mg, optionally 250 mg.

10. The mitragynine composition of claim 8, wherein the composition is in a dosage form of a gelatin- or vegetarian capsule, and wherein the size of the capsule can be 1, 2, 3, 4, or 5, optionally size 1.

11. The mitragynine composition of claim 8, wherein the composition comprises 5 mg to 40 mg mitragynine, in particular 5 mg to 20 mg mitragynine, and in more particular about 10 mg mitragynine.

12. The mitragynine composition of claim 8, wherein the composition further comprises 10% to 50% (w/w) crospovidone, optionally about 40% (w/w) crospovidone.

13. The mitragynine composition of claim 8, wherein the remaining weight is filled with an excipient, wherein the excipient is, optionally, microcrystalline cellulose.

14. The mitragynine composition of claim 8, wherein the composition may comprise further excipients.

15. A method of treatment for osteoarthritis in a subject canine, comprising administering the kratom composition of claim 1 or mitragynine composition of claim 8 at a dose of 0.2 mg/kg to 1.0 mg/kg, in particular 0.3 mg/kg to 0.7 mg/kg, and in more particular 0.5 mg/kg to 0.7 mg/kg mitragynine to the subject.

16. The method of claim 12, wherein the kratom composition of claim 1 or mitragynine composition of claim 8 is administered to the subject every 8-12 hour for 3-5 weeks.

17. A method of treatment for post-surgical pain in a subject canine, comprising administering the kratom composition of claim 1 or mitragynine composition of claim 8 at a dose of 0.2 mg/kg to 1.0 mg/kg, in particular 0.3 mg/kg to 0.7 mg/kg, and in more particular 0.5 mg/kg to 0.7 mg/kg mitragynine to the subject.

18. The method of claim 14, wherein the kratom composition of claim 1 or mitragynine composition of claim 8 is administered 1 hour before surgery and continued every 8-12 hours for 5-10 days after surgery.

19. A kratom formulation comprising:
(a) about 5% to about 30% by weight kratom extract powder, optionally about 6.5% by weight kratom extract powder;
(b) about 5% to about 25% by weight polyanionic beta cyclodextrin derivative, optionally about 6.5% by weight polyanionic beta cyclodextrin derivative;
(c) about 12% to about 48% by weight crospovidone, optionally about 12% by weight crospovidone;
(d) about 0% to about 75% by weight microcrystalline cellulose, optionally about 75% by weight microcrystalline cellulose; and
(e) optionally a gelatin or veg capsule.
